(19) United States
(12) Patent Application Publication
(10) Pub. No.: US 2017/0107564 A1
(43) Pub. Date: Apr. 20, 2017

(54) COMPOSITIONS FOR QUANTITATIVE AND/OR SEMI-QUANTITATIVE MUTATION DETECTION METHODS

(71) Applicant: VELA OPERATIONS SINGAPORE PTE. LTD., Singapore (SG)

(72) Inventors: Mengchu WU, Singapore (SG); Arseny SMIRNOV, Singapore (SG)

(21) Appl. No.: 15/317,775

(22) PCT Filed: Jun. 30, 2015
(86) PCT No.: PCT/IB2015/001085

§ 371 Date: Dec. 9, 2016

(30) Foreign Application Priority Data
Jun. 30, 2014 (GB) 1411603.2

(51) Int. Cl. C12Q 1/68

(52) U.S. Cl. CPC C12Q 1/6827

(57) ABSTRACT

The present invention relates to compositions which may be used as controls and/or references for quantitative and/or semi-quantitative detection methods, in particular for digital PCR or next generation sequencing assays. The present invention also describes synthetic nucleic acid constructs, in particular plasmids which are present in said compositions, kits, their uses and a method involving the use of the compositions according to the present invention. Furthermore, methods for providing the compositions according to the present invention are described herein.

### FIELD OF THE INVENTION

The present invention relates to compositions which may be used as controls and/or references for quantitative and/or semi-quantitative mutation detection methods, in particular for digital PCR or next generation sequencing methods. The present invention also describes synthetic nucleic acid constructs, in particular plasmids which are present in said compositions, kits comprising the composition, their uses and a method involving the use of the compositions according to the present invention. Furthermore, a method for providing the compositions according to the present invention is described herein.

### BACKGROUND OF THE INVENTION

The use of nucleic acid sequencing has become an essential tool in many diagnostic areas in modern medicine. In particular, Next Generation Sequencing (NGS)-based genetic tests are quickly gaining acceptance in clinical diagnostics. An example of such an area is oncology, where nucleic acid sequencing is employed in order to identify whether e.g. oncogenic mutations are present in a gene or whether cancer-inducing and/or indicating translocations are present in a genome. Further, nucleic acid sequencing is employed to detect whether a pathogenic microorganism (such as e.g. a bacteria or a virus) is present in a clinical sample, e.g. a tissue sample or a blood sample from a human patient. In the latter method, nucleic acid sequences are detected, which are not found in a human subject but only in the microorganism. NGS methods thus may result in the identification and sequence determination of a target sequence, which may indicate the presence or absence of an oncogenic mutation or the presence or absence of a pathogen-derived nucleic acid.

Thus, diagnostic kits and/or methods based on NGS techniques and other quantitative and/or semi-quantitative mutation detection methods are presently developed for diagnostic purposes. However, due to regulatory provisions, in many countries the accuracy and usefulness of such diagnostic kits and methods has to be shown (e.g. by meeting a certain sensitivity threshold) before an actual product is admitted to the market.

In this context it is often required to show that the method used is suitable in detecting rare mutations or rare nucleic acids, e.g. mutations or nucleic acids occurring a clinical sample at a frequency of 5% or less. Additionally, in order to ensure that the diagnostic method has been performed properly, thus avoiding any false negative results, it is also necessary to provide positive controls carrying the mutation(s) or nucleic acids to be detected at a similar low frequency as usually present in the clinical sample.

However, for rare mutations such as mutations occurring in certain types of cancer or for nucleic acids which occur in a sample at a low percentage (e.g. nucleic acids derived from pathogens), it is extremely difficult to obtain clinical samples carrying the mutation and/or the nucleic acid to be detected at the required frequency which may be used as testing material. Hence, there is a need for reference compositions and/or control compositions comprising rare mutations and/or the nucleic acid to be detected at the required frequency.

### OBJECT AND SUMMARY OF THE INVENTION

It is thus an object of the present invention to provide such reference and/or control compositions.

In the context of the present invention it now has been surprisingly found that compositions comprising (i) synthetic nucleic acid constructs, in particular plasmids comprising at least one inserted wild-type nucleic acid sequence or (ii) genomic DNA and (iii) synthetic nucleic acid constructs, in particular plasmids comprising at least one inserted target nucleic acid sequence at a defined molar ratio may be used as such reference and/or control compositions for semi-quantitative and/or quantitative methods such as NGS or digital PCR.

By using the compositions of the present invention there is no longer the need to use clinical samples which may be hard to obtain and may vary in the frequency in which the mutations or nucleic acids to be detected occur therein. Furthermore, the compositions of the invention provide the advantage that they may be provided for any desired mutation or nucleic acid to be detected or combination of mutations or nucleic acids to be detected and for any desired percentage of mutations or nucleic acids to be detected.

In the following description reference is made to plasmids (i) and (iii) which are present in the composition according to the invention. It is however to be understood that of course any other synthetic nucleic acid construct capable of comprising either a wild-type target sequence or a mutant thereof or any other sequence, such as a sequence derived from a pathogen may also be used in the context of the present invention.

Hence, in one aspect the present invention relates to a composition comprising:

(i) plasmids comprising at least one inserted wild-type nucleic acid sequence or
(ii) a wild-type genomic DNA sequence in a defined molar ratio with
(iii) plasmids comprising at least one inserted target nucleic acid sequence.

One embodiment relates to the composition, wherein the plasmids (iii) comprise 1-50 inserted target nucleic acid sequence(s). In another embodiment, the plasmids (iii) comprise 1-30 inserted target nucleic acid sequence(s).

In a further embodiment the plasmids (iii) comprise 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 inserted target nucleic acid sequence(s).

In yet another embodiment, the defined molar ration of (iii) to (i) or (ii) is in the range of 1:10-1:30, optionally the defined molar ration of (iii) to (i) or (ii) is 1:15 to 1:25, e.g. 1:20, 1:18, optionally 1:19.

In another embodiment, the composition is a positive control composition or a reference composition for quantitative and/or semi-quantitative mutation detection methods.

In one embodiment, the quantitative and/or semi-quantitative mutation detection method is digital PCR and/or next generation sequencing (NGS).

Another aspect of the invention relates to the use of a composition as described herein as a control composition and/or as a reference composition in quantitative and/or semi-quantitative mutation detection methods. In one embodiment, the composition is used as a positive control composition.

In another aspect, the composition is used to test the sensitivity of a quantitative and/or semi-quantitative mutation detection method.

In another embodiment, sensitivity for one or more target sequence(s) occurring at 10%, 9%, 8%, 7%, 6%, 5%, 4% or 3% on average in the sample is to be confirmed.

In a further embodiment, the mutation detection method is digital PCR and/or next generation sequencing (NGS).

In another embodiment, the quantitative and/or semi-quantitative mutation detection method is for detection of the presence or absence of sequences derived from a pathogen or an oncogene.

A further aspect of the present invention relates to a method of confirming the sensitivity of a semi-quantitative and/or quantitative detection method for one or more target sequence(s) comprising the following steps:
(i) providing a composition as described herein,
(ii) performing the semi-quantitative and/or quantitative detection method using said composition, and
(iii) assessing the sensitivity of the detection method.

Yet another aspect of the present invention relates to a method for preparation of a composition as described herein, wherein the method comprises the following steps:
(i) introduction of the wild-type sequence into a plasmid or provision of a wild type genomic DNA (gDNA),
(ii) introduction of at least one target sequence into a separate plasmid,
(iii) absolute quantification of the plasmids and/or the gDNA
(iv) preparing a composition of (i) and (ii) at a defined molar ratio.

In one embodiment of the method for preparation of the composition as described herein, the absolute quantification of the plasmids or the gDNA is performed by digital PCR (dPCR), optionally by digital droplet PCR (ddPCR).

Another aspect of the invention relates to a kit comprising the composition as described herein.

### BRIEF DESCRIPTION OF THE FIGURE

FIG. 1 depicts the list of plasmid compositions prepared as testing material for the NGS assay as used as in the below described example.

### DEFINITIONS

As used in the specification and the claims, the singular forms of “a” and “an” also include the corresponding plurals unless the context clearly dictates otherwise. Vice versa, when the plural form of a noun is used, it also refers to the singular form unless the context clearly dictates otherwise. For example, when mutations are mentioned, this is also to be understood as relating to a single mutation.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

Furthermore, the terms first, second, third or (i), (ii), (iii) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. However, in a specific embodiment of the invention, the method steps (i), (ii) and (iii), optionally including any intermediate steps defined herein, are performed in chronological order.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

In the context of the present invention the term “nucleic acid” refers to a naturally occurring deoxyribonucleotide or ribonucleotide polymer in either single- or double-stranded form. The nucleic acid may particularly be double-stranded DNA and single-stranded RNA.

The term “sequence” as used herein refers to the sequential occurrence of the bases in a deoxyribonucleotide or ribonucleotide polymer, wherein a base found in a deoxyribonucleotide polymer is selected from the group consisting of A, T, G and C and a base found in a ribonucleotide polymer is selected from the group consisting of A, U, G and C. A sequence of bases in a deoxyribonucleotide polymer may thus e.g. be GGAAGCAAGCCT, whereas a sequence of bases in a ribonucleotide polymer may e.g. be GGAAUCGAU.

As used herein “wild-type sequence” or “wild-type nucleic acid sequence” relates to the nucleic acid sequence which usually occurs in a population, e.g. in humans. In the context of the present invention “wild-type sequences” denote said sequences, which are not indicative for a disorder (e.g. a certain type of cancer) or pathogenic organism to be detected by the diagnostic method used. The same holds true for “wild-type genomic DNA” as used herein, which also relates to said DNA sequence which is normally occurs in a population (e.g. in humans) or, as used in the context of the present invention, said DNA sequence which is not indicative for a disorder or a pathogenic organism to be detected by the diagnostic method used.

A “target sequence” or “target nucleic acid sequence” as referred to herein is a nucleic acid sequence, the presence or absence of which is detected in the methods according to the present invention and which is indicative for a disorder or a pathogenic organism to be detected by the diagnostic method used. The presence of one or more target sequence(s) may be indicative for cancer (e.g. an oncogene) or the presence of a microorganism, in particular a pathogenic microorganism, or a nucleic acid sequence that is implicated in the development, severity, recovery, etc., from any other disease, e.g. a metabolic disease, hereditary disorder, autoimmune disease, and the like.

Target nucleic acids include but are not limited to DNA such as but not limited to genomic DNA, mitochondrial DNA, cDNA and the like, and RNA such as but not limited to mRNA, miRNA, and the like. The target nucleic acid may derive from any source including naturally occurring sources or synthetic sources. The nucleic acids may be PCR products, cosmids, plasmids, naturally occurring or synthetic library members or species, and the like. The invention is not intended to be limited in this regard. The nucleic acid may be from animal or pathogen sources including without limitation mammals such as humans, and microbes such as bacteria, viruses, fungi, parasites, and mycobacteria. In some embodiments, the nucleic acid is not a viral nucleic acid. The target nucleic acid can be obtained from any bodily fluid or tissue including but not limited to blood, saliva, cerebrospinal fluid (“CSF”), skin, hair, urine, stool, and mucus. The target nucleic acid may also be derived from without limitation an environmental sample (such as a water sample), a food sample, or a forensic sample, the sample may be a fresh sample (e.g. biopsy material directly subjected to nucleic acid extraction), or a sample that has been treated to allow storage, e.g. a sample that was formalin-fixed and/or paraffin-embedded (FFPE samples).

In particular, the target sequence may be a sequence carrying a mutation of the wild-type sequence previously inserted into plasmid (i) or the wild-type genomic DNA (ii) which should be detected with diagnostic methods as described herein (i.e. a target mutation). These target sequences carrying (a) target mutation(s) may be indicative for pathogenic conditions such as cancer (i.e. so-called oncogenes). Furthermore, a target sequence may be a sequence indicative for the presence of a pathogene, such as the presence of a pathogenic microorganism.

“Rare mutation” or “rare nucleic acid sequence” as used herein denotes any mutation or sequence of nucleic acids which is present in a clinical sample obtained from a patient at a less than 10% on average, in particular at less than 5% on average. “Target sequence present in a sample at x % on average” denotes the percentage x at which the target sequence is present in the sample in relation to the amount of wild-type sequence present in the sample. Hence, the percentage of the target sequence as used herein always relates to the ratio between the amount of the wild-type sequence and the amount of the target sequence present in the composition and/or sample.

As used herein, the term “sample” refers to any biological sample from any human or veterinary subject that may be tested for the presence of a nucleic acid comprising a target sequence. The samples may include tissues obtained from any organ, such as for example, lung tissue and fluids obtained from any organ such as for example, blood, plasma, serum, lymphatic fluid, synovial fluid, cerebrospinal fluid, amniotic fluid, amniotic cord blood, tears, saliva, and nasopharyngeal washes. As listed above, samples may also be derived from a specific region in the body, e.g. the respiratory tract; samples from the respiratory tract include throat swabs, throat washings, nasal swabs, and specimens from the lower respiratory tract. Samples as used herein also include solid tissue samples comprising tumour tissue. These samples may comprise tumour tissue and the surrounding tissue or tumour tissue only.

The sample may be derived from a human or a veterinary subject. Accordingly, a “patient” may be a human or veterinary subject. If reference is made to a “clinical sample”, this indicates that the sample is from a patient suspicious of carrying a nucleic acid comprising a target sequence.

The term “oncogene” is used herein in its common meaning in molecular biology and oncology, respectively. Thus, there are e.g. mutations known in genes, which render a “normal or wild-type” gene oncogenic, i.e. cancer-inducing; examples in this respect are mutations rendering kinases constitutionally active such that specific signals (e.g. growth inducing signals) are constantly transmitted and corresponding processes initiated. “Oncogenes” as used herein may also relate to intra- or inter-chromosomal translocations resulting also in cancer-inducing situations.

The term “microorganism” as used herein is used in its broadest meaning. Thus, a microorganism may be any type of bacteria, archaeum, protozoum, fungus and virus. It is explicitly mentioned that viruses fall under the definition of a “microorganism” as used herein. The term “pathogen” or “pathogenic microorganism” as used herein relates to any type of microorganism having the capacity to cause a disease in a patient.

The term “detecting the presence” as used herein is to be understood in the meaning of “detecting the presence or absence”.

“Diagnostic method” as used herein denotes any quantitative or semi-quantitative mutation detection method which may be used for diagnostic purposes, e.g. for detection of a target sequence present in a sample obtained from a patient. In particular, diagnostic method denotes next generation sequencing methods and droplet PCR (dPCR), optionally digital droplet PCR (ddPCR).

“Quantitative detection method” or “quantitative mutation detection method” denotes any detection method which may be used to determine the quantity of a target sequence such as next generation sequencing, qPCR or digital PCR. “Semi-quantitative detection method” or “semi-quantitative mutation detection method” as used herein relates to any method allowing approximating the quantity of a target sequence such as PCR or RT-PCR.

“Digital PCR” as used herein relates to any PCR method in which the sample is partitioned into a large number of small sub-samples which subsequently are each subjected to a PCR amplification reaction. After PCR amplification, the nucleic acids may be quantified by counting the sub-samples that contain PCR end-product (positive reactions) and the sub-samples containing no PCR end-product (negative reactions) taking into account the Poisson distribution. Digital PCR (dPCR) is, contrary to conventional PCR, not dependent on the number of amplification cycles performed in order to allow for a determination of the initial sample amount, thus eliminating the reliance on uncertain exponential data to quantify target nucleic acids and providing absolute quantification. “Digital droplet PCR” as used herein relates to a digital PCR method in which the initial sample is sub-divided into several droplets constituting the sub-samples.

As used herein, the term “next generation sequencing” or “next generation sequencing method” refers to any sequencing technology having an increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization.

As used herein, the term “amplification” refers to enzyme-mediated procedures that are capable of producing billions of copies of nucleic acid target. Examples of enzyme-mediated target amplification procedures known in the art include PCR.

“Extracting nucleic acids” means that any nucleic acids present in a vial are isolated from any cellular background, particularly isolated from intact cells or tissues. Preferably, the nucleic acids are also washed during the process and optionally concentrated. Following extraction, all cellular or tissue debris not related to nucleic acids has been removed. Typical extraction methods may include the use of hypotonic lysis buffer, heat and/or detergents, and are known to the skilled person.

The term “sequencing” is used herein in its common meaning in molecular biology. Thus, the exact sequential occurrence of bases in a nucleic acid sequence is determined.

### DETAILED DESCRIPTION OF THE INVENTION

As discussed above, there is a need for compositions which may be used as a control or reference composition comprising rare mutations or rare nucleic acids to be detected in semi-quantitative or quantitative nucleic acid mutation detection methods.

In the context of the present invention it now has been found that a composition comprising plasmids comprising inserted wild-type nucleic acid sequences or wild-type genomic DNA in a defined molar ratio with plasmids comprising at least one inserted target sequence may be used for such purposes.

Thus, one aspect of the present invention relates to a composition comprising:

(i) plasmids comprising at least one inserted wild-type nucleic acid sequence or
(ii) a wild-type genomic DNA sequence in a defined molar ratio with
(iii) plasmids comprising at least one inserted target nucleic acid sequence.

The inserted target nucleic acid sequence may be any sequence which carries a mutation of the wild-type sequence or a sequence indicative for the presence of a pathogen to be detected by a diagnostic method as described herein. Mutations to be detected by the diagnostic methods as described herein may be any mutation(s) which is/are indicative for a disease or a disorder such as cancer.

In one embodiment, the plasmids (iii) may comprise any target sequence or combination of target sequences indicative for cancer. For example, the target sequence or combination of target sequences may be indicative for lung cancer, breast cancer, thyroid cancer, leukemia, melanoma, colorectal cancer, prostate cancer, liver cancer, lymphoma or ovarian cancer. In one embodiment, the target sequence or combination of target sequences is indicative for melanoma, non small cell lung cancer, colorectal cancer, thyroid cancer and/or leukemia. In one embodiment, the target sequence or combination of target sequences is indicative for melanoma. In one embodiment, the target sequence or combination of target sequences is indicative for non small cell lung cancer. In one embodiment, the target sequence or combination of target sequences is indicative for colorectal cancer. In one embodiment, the target sequence or combination of target sequences is indicative for thyroid cancer. In one embodiment, the target sequence or combination of target sequences is indicative for leukemia.

In one embodiment, the target sequence or combination of target sequences is indicative for an oncogene, optionally a human oncogene. The human oncogene to be detected may be a mutation in a gene selected from the group consisting of BRAF, NRAS, CDKN2A, MAP2K1, MAP2K2, FGFR3, FGFR4, AKT3, KIT, PIK3CA, GNA11 and GNAQ. In one embodiment, the target sequence or combination of target sequences to be detected is/are one or more mutations selected from the group shown in Table 1 below:

| Gene | Mutation |
|---|---|
| BRAF | 1742A > G, 1756G > A, 1774A > G, |
| | 1789C > G, 1803A > C, 1760A > C, |
| | 1776A > G, 1789_1790CT > TC, 1803A > T, |
| | 1761C > G, 1781A > T, 1796C > T, |
| | 1813_1814AG > TT, 1761C > A, 1781A > G, |
| | 1797_1799AGT > GAG, 1814G > A, |
| | 1782T > A, 1798_1799GT > AA, 1783T > C, |
| | 1798_1799GT > AG, 1784T > C, 1798G > A, |
| | 1785T > G, 1798G > T, 1785T > A, |
| | 1799_1800TG > AA, 1786G > C, 1799T > A, |
| | 1790T > G, 1790T > A, |
| | 1797_1797A > TACTACG, |
| | 1799_1800TG > AT, 1799T > G, 1799T > C, |
| | 1801A > G, 1801_1803delAAA |
| NRAS | 180_181AC > TA, 34G > C, 52G > A, |
| | 181_182CA > AG, 34G > T, 181C > A, |
| | 34G > A, 181C > G, 35G > A, |
| | 181_182CA > TT, 35G > C, |
| | 181_183CAA > AAG, 35G > T, 182A > C, |
| | 37G > C, 182A > T, 37G > T, 182A > G, |
| | 37G > A, 182_183AA > TG, 38_39GT > TC, |
| | 182_183AA > GG, 38G > A, 183A > T, |
| | 38G > T, 183A > C, 383G > C |
| CDKN2A | 171_172CC > TT, 205G > T, 172C > T, |
| | 181G > T |
| MAP2K1 | 332T > G, 362G > C, 370C > T |
| MAP2K2 | 361T > A |
| FGFR3 | 742C > T, 1138G > A, 1172C > A, 1921G > A, |
| | 746C > G, 1948A > C, 1949A > T, 1949A > C |
| FGFR4 | 1948A > G |
| AKT3 | 511G > A, 371A > T |
| KIT | 154G > A, 1727T > C, 1755C > T, 1924A > G, |
| | 1961T > C, 2446G > T, 2466T > A, |
| | 1651_1665del15, 1656_1673del18, |
| | 1735_1737delGAT, 2446G > C, 2466T > G, |
| | 1667_1672delAGTGGA, |
| | 1669_1674delTGGAAG, 2447A > T, |
| | 2467T > G, 1669T > A, 2474T > C, 1669T > C, |
| | 1669T > G, 1669_1683del15, |
| | 1672_1680del9, 1675_1677delGTT, |
| | 1672_1686del15, 1673_1687del15, |
| | 1675G > A, 1676T > G, 1676T > C, 1679T > A, |
| | 1679T > G |
| PIK3CA | 1616C > G, 1633G > A, 1624G > A, |
| | 1637A > C, 1624G > C, 1637A > G, |
| | 1625A > T, 1633G > C, 1634A > G, |
| | 1634A > C, 1635G > C, 1636C > A, |
| | 1636C > G, 1637A > T |
| GNA11 | 626A > T |
| GNAQ | 626A > T |

However, in another embodiment, the target sequence or combination of target sequences may also be indicative for the presence of a microorganism, in particular the presence of a pathogenic microorganism. Thus, the target sequence or combination of target sequences may also be indicative for the presence of viruses, for example the hepatitis C virus (HCV) or human immunodeficiency virus (HIV).

In another embodiment, the target sequences or combinations of target sequences indicative for the presence of an oncogene and a microorganism are present on plasmids (iii).

In one embodiment of the invention, the individual plasmids (iii) comprise more than one inserted target sequence, e.g. 1-50 target sequence(s). In another embodiment, the plasmids (iii) comprise 1-40 target sequence(s). In yet another embodiment, the plasmids (iii) comprise 1-30 target sequence(s). In a further embodiment, the plasmids (iii) comprise 1-20 target sequence(s). In another embodiment, the plasmids (iii) comprise 1-10 target sequence(s). In a further embodiment, the plasmids (iii) comprise 1-5 target sequence(s).

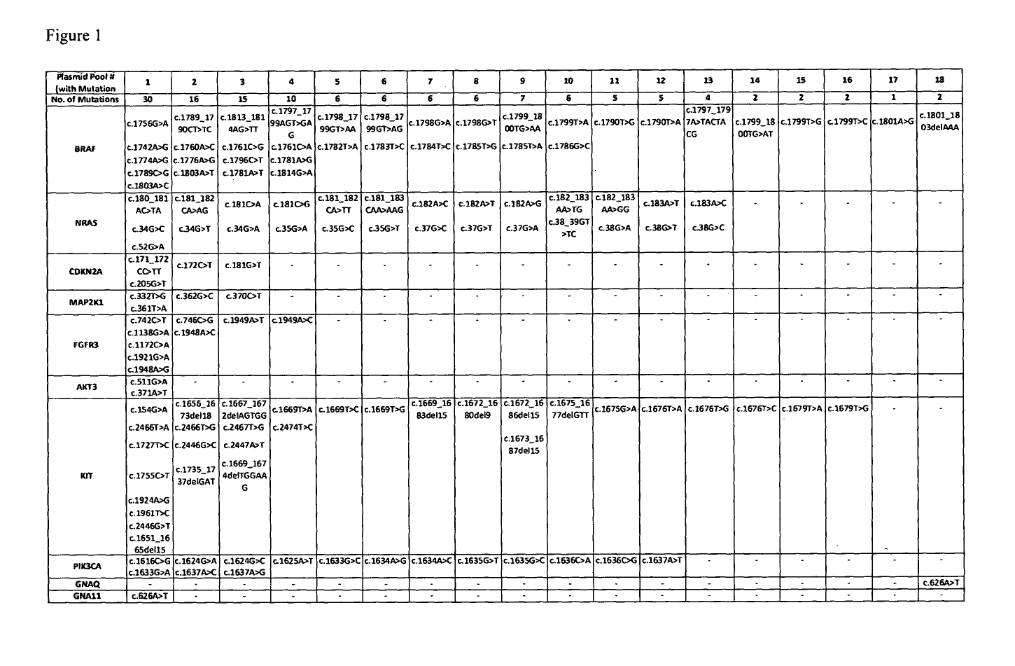

In yet a further embodiment of the invention, the plasmids (iii) may comprise any of the combinations of target sequences depicted in FIG. 1. In one embodiment, the plasmids (iii) comprise the combination of target sequences of plasmid pool No. 1 of FIG. 1. In one embodiment, the plasmids (iii) comprise the combination of target sequences of plasmid pool No. 2 of FIG. 1. In one embodiment, the plasmids (iii) comprise the combination of target sequences of plasmid pool No. 3 of FIG. 1. In one embodiment, the plasmids (iii) comprise the combination of target sequences of plasmid pool No. 4 of FIG. 1. In one embodiment, the plasmids (iii) comprise the combination of target sequences of plasmid pool No. 5 of FIG. 1. In one embodiment, the plasmids (iii) comprise the combination of target sequences of plasmid pool No. 6 of FIG. 1. In one embodiment, the plasmids (iii) comprise the combination of target sequences of plasmid pool No. 7 of FIG. 1. In one embodiment, the plasmids (iii) comprise the combination of target sequences of plasmid pool No. 8 of FIG. 1. In one embodiment, the plasmids (iii) comprise the combination of target sequences of plasmid pool No. 9 of FIG. 1. In one embodiment, the plasmids (iii) comprise the combination of target sequences of plasmid pool No. 10 of FIG. 1. In one embodiment, the plasmids (iii) comprise the combination of target sequences of plasmid pool No. 11 of FIG. 1. In one embodiment, the plasmids (iii) comprise the combination of target sequences of plasmid pool No. 12 of FIG. 1. In one embodiment, the plasmids (iii) comprise the combination of target sequences of plasmid pool No. 13 of FIG. 1. In one embodiment, the plasmids (iii) comprise the combination of target sequences of plasmid pool No. 14 of FIG. 1. In one embodiment, the plasmids (iii) comprise the combination of target sequences of plasmid pool No. 15 of FIG. 1. In one embodiment, the plasmids (iii) comprise the combination of target sequences of plasmid pool No. 16 of FIG. 1. In one embodiment, the plasmids (iii) comprise the target sequence of plasmid pool No. 17 of FIG. 1. In one embodiment, the plasmids (iii) comprise the target sequence of plasmid pool No. 18 of FIG. 1. Of course, it is also possible to combine one or more of the pools referred to above as desired.

It is to be understood that plasmids (i) may comprise the wild-type sequences corresponding to the target sequences of plasmids (iii) and, consequently, also the same amount of wild-type sequences as target sequences inserted in plasmids (iii). For example, if plasmids (iii) comprise five target sequences having mutations in the BRAF gene, plasmids (i) may also comprise the corresponding five wild-type sequences of the BRAF gene. In general, the plasmids (i) and (iii) may carry all desired sequences (wild-type or mutant sequences) on one or more different individual plasmids or sub-pools. For example, a plasmid pool comprising about 30 inserted sequences may carry all of these on single plasmid constituting the plasmid pool. Alternatively, it is also contemplated that the plasmid pools comprises more than one type of plasmid, e.g. 2, 3, 4, 5, or more plasmids that carry several, e.g. 3, 4, 5 or more inserted sequences. These individual plasmids form subgroups that may together form a pool of plasmids. It is an advantage of the present invention that subgroups of plasmids may be used for the validation of different assays, e.g. when the target genes can be used in different assays, e.g., cancer assay 1 and cancer assay 2, without the to redesign entire plasmid pools (i) and (iii).

If more than one target sequence or more than one wild-type sequence is inserted into the plasmid (i) or (iii), the wild-type or target sequences may be introduced in a certain distance into the plasmid. The distance between the wild-type or target sequences may any distance considered suitable by the person skilled in the art. It is to be understood that the distances between the different wild-type or target sequences present on plasmids (iii) may be the same between each of the wild-type or target sequences or may vary between the different wild-type or target sequences. The distance between the wild-type or target sequences present on plasmid (iii) may be at least 10 bp, at least 30 bp, at least 50 bp, at least 100 bp, at least 150 bp, at least 200 bp, at least 250 bp, at least 300 bp, at least 350 bp or at least 400 bp.

The plasmids present in the composition may be any plasmids considered suitable by the person skilled in the art for such a purpose. Suitable plasmids for such a purpose are known to the person skilled in the art and may include pBR322, pBR327, pUC-8, pUC-19, pUC-57, pGEM3Z, Ml3mp1, M13mp2 and M13mp7.

If the composition of the present invention comprises (i) plasmids comprising at least one inserted wild-type sequence and (iii) plasmids comprising at least one inserted target sequence, plasmids (i) and (iii) may be based on the same type of plasmid or on different types of plasmids. In one embodiment, plasmids (i) and (iii) are based on the same type of plasmid. In another embodiment, plasmids (i) and (iii) are both based on pUC-57.

Plasmid (i) or gDNA (ii) may be present in any molar ratio with plasmid (iii) considered suitable by the person skilled in the art to achieve and/or confirm the desired sensitivity of the diagnostic method. The desired sensitivity may be any sensitivity which the diagnostic method has to achieve, e.g. for marketing authorization purposes. “Sensitivity” as used herein denotes the proportion of target sequences which are actually correctly identified as such by the diagnostic method, i.e. it relates to the ability of the diagnostic method to identify the target sequences correctly.

The molar ratio of (iii) to (i) or (ii) may be any molar ratio allowing to achieve the average percentage at which the target sequence which should be detected by the diagnostic test normally occurs in a sample, in particular in a clinical sample. The molar ratio of (iii) to (i) or (ii) may be any molar ratio at which the target sequence is present at about 10%, 9%, 8%, 7%, 6%, 5%, 4%, 3%, 2% or 1% in the composition according to the invention. In one embodiment, the molar ratio of (iii) to (i) or (ii) is a molar ratio at which the target sequence is present at about 5% in the composition according to the invention.

Accordingly, the molar ratio of (iii) to (i) or (ii) may be in the range from 1:10-1:30. In one embodiment, the molar ratio of (iii) to (i) or (ii) is selected from the group consisting of 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29 and 1:30. In one embodiment of the invention, the molar ratio of (iii) to (i) or (ii) is 1:19. In the context of the present invention it has been found that when the molar ratio of (iii) to (i) or (ii) is 1:19, the percentage of the target nucleic acid in the composition is about 5%.

The compositions as described herein may also be introduced into cells or cell lines, which are, e.g., subsequently formalin-fixed and paraffin-embedded. Hence a formalin-fixed and paraffin-embedded (FFPE) reference composition and/or control composition may be provided. Such a FFPE composition may in particular be suitable when the samples which are to be tested by the diagnostic method are typically FFPE samples, since the FFPE reference and/or control composition may better reflect the background present in the sample obtained from a human or veterinary subject. Hence, another aspect of the present invention relates compositions as described herein which have been introduced into cells which are subsequently formalin-fixed and paraffin-embedded.

The composition described herein may be used as reference or control composition for any quantitative or semi-quantitative method. However, in one aspect of the invention, the composition as described herein is used as a reference or control composition in a NGS method or digital PCR. In a specific embodiment, the detection method in which the composition described herein is used as reference or control composition is a NGS method. In yet another embodiment, the detection method in which the composition described herein is used as a reference or control composition is digital PCR, optionally digital droplet PCR.

Reference compositions are typically used in order to confirm and/or test the sensitivity of a diagnostic method (e.g. a quantitative and/or semi-quantitative mutation detection method), while control compositions are used in order to ensure that the diagnostic method (e.g. the quantitative and/or semi-quantitative method) was correctly performed. The composition described herein may be used for both purposes.

“Reference compositions” (or “standards”) in the context of the present invention are compositions comprising one or more target sequence(s) (such as a sequence comprising a rare mutation or a sequence specific for a certain pathogen) in a predetermined amount, which may be used for testing if the diagnostic method is able to detect and/or quantitate the target sequence. “Control compositions” as used in the context of the present invention denotes any composition which is used as a control, in particular a positive control, in order to ensure that the diagnostic method has been performed properly. Typically, control compositions comprise the one or more target sequences which should be detected by the diagnostic method in a predetermined amount. Hence, in one embodiment of the invention the control composition is a positive control composition. The predetermined amount in which the target sequence is present in the reference composition or control composition typically reflects the average percentage at which the target sequence(s) is present in a sample to be tested for the presence or absence of the target sequence. However, in particular with respect to reference compositions, the predetermined amount may also be any percentage of target nucleic acid present in the sample which, e.g. for marketing authorization purposes, the diagnostic method should be able to detect. Different percentages of the target sequences and corresponding molar ratios are described above. Thus, in one embodiment of the reference composition it comprises about 5% target sequence(s).

In another aspect of the invention, the composition as described herein can be used to confirm the sensitivity of a diagnostic method (e.g. of a quantitative and/or semi-quantitative mutation detection method).

In particular, the composition as described herein may be used in order to confirm the sensitivity of the diagnostic method for one or more target sequence(s) which is/are present at about 10%, 9%, 8%, 7%, 6%, 5%, 4%, 3%, 2% or 1% on average in the sample derived from a human or veterinary subject. In one embodiment of the invention, the composition as described herein is used to confirm the sensitivity of a diagnostic method for one or more target sequence(s) which is/are present at 10% on average in a sample derived from a human or veterinary subject. In yet another embodiment of the invention, the composition as described herein is used to confirm the sensitivity of the diagnostic method for one or more target sequence(s) which is/are present at about 5% on average in a sample derived from a human or veterinary subject.

In one embodiment of the invention, the composition as described herein can be used to confirm the sensitivity of a digital PCR and/or a next generation sequencing method. In one embodiment, the composition is used to confirm the sensitivity of a digital PCR, in particular a digital droplet PCR. In yet another embodiment, the composition is used to confirm the sensitivity of a next generation sequencing method.

In a further embodiment, the diagnostic method for which the sensitivity is to be confirmed is a quantitative and/or semi-quantitative mutation detection method for detection of the presence or absence of target sequences such as target sequences indicative for an oncogene or a pathogenic organism as described herein.

Another aspect of the invention relates to a method of confirming the sensitivity of a diagnostic method such as a semi-quantitative and/or quantitative mutation detection method, wherein said method comprises the following steps:

(i) providing a composition as described herein,
(ii) performing the detection method using said composition, and
(iii) assessing the sensitivity of the method.

Step (i) of said method may include any step of the method for preparation of the composition described herein. It is to be understood that the composition may be adapted for the purpose for which it is intended to be used. If, for example, the sensitivity of the diagnostic method for one or more target sequence(s) which is/are present at 5% on average in a sample should be confirmed, a reference composition comprising the target sequence at 5% has to be used. Thus, in one embodiment, a composition comprising plasmids (iii) and plasmids (i) or gDNA (ii) at a molar ratio of 1:19 is used in the method of confirming the sensitivity of a diagnostic method.

In step (ii) the detection method (e.g. a semi-quantitative and/or quantitative detection method) for which the sensitivity should be confirmed may be performed by using the composition as described herein as a reference composition. For instance, in step (ii) a next generation sequencing method or a digital PCR is performed using a composition as described herein as reference composition. In one embodiment of the method, in step (ii) a next generation sequencing method is performed. In another embodiment of the method, in step (ii) a digital PCR, in particular a digital droplet PCR, is performed.

In step (iii) of the method of confirming the sensitivity of a diagnostic method as described herein, the sensitivity of the diagnostic method for detection of the target sequence is assessed. Since the percentage of target sequence which is present in the reference composition used is known, this can be done by comparing the results achieved by the detection method performed with the known percentage of target sequences present in the reference composition.

It is to be understood that steps (i)-(ii) may be performed in several parallel runs and, subsequently, in step (iii) the average percentage of the values obtained in the runs is compared with the known percentage of the target nucleic acid which is present in the reference composition.

Hence, in one embodiment steps (i)-(ii) may be performed in at least 2, at least 3, at least 5, at least 10, at least 15, at least 20, at least 25, at least 30, at least 50 or at least 100 parallel runs and the average percentage of target nucleic acids detected in said runs is subsequently compared with the known percentage of target nucleic acid present in the reference composition. In another embodiment steps (i)-(ii) are performed in 2-100 parallel runs, optionally in 10-50 parallel runs and the average percentage of target nucleic acids detected in said runs is subsequently compared with the known percentage of target nucleic acid present in the reference composition. In another embodiment steps (i)-(ii) are be performed in 2, 3, 5, 10, 15, 20, 25, 30, 50 or 100 parallel runs and the average percentage of target nucleic acids detected in said runs is subsequently compared with the known percentage of target nucleic acid present in the reference composition.

If in step (iii) it is found that the percentage of the target sequence determined by the diagnostic method to be tested corresponds to the known percentage of target nucleic acid present in the reference composition, the sensitivity of the test for the reference composition is confirmed. It is to be understood that as used in the context of the present invention “corresponding to the known percentage of target nucleic acid present in the reference composition” does not necessarily mean that the percentage of the target sequence determined by the diagnostic method to be tested and the percentage present in the reference composition do have to be exactly the same value. It is to be understood by the person skilled in the art that certain deviations between these values may be accepted, in particular, if the average value of parallel runs which have been performed is determined. The amount of deviation between the different values may depend on the required sensitivity of the test. However, in one embodiment, a deviation between the value of target sequence determined by the diagnostic method to be tested and the known value of target sequence present in the reference composition of 0.1%-10%, optionally, of 0.1-5% may be considered to be acceptable. In one embodiment, a deviation between the value of target sequence determined by the diagnostic method to be tested and the known value of target sequence present in the reference composition of 0.1%, 0.2%, 0.3%, 0.4%, 0.5%, 0.7%, 0.8%, 0.9%, 1.0%, 1.5%, 2.0%, 2.5%, 3.0%, 3.5%, 4.0%, 4.5% or 5% may be considered to be acceptable in order to confirm the sensitivity of the diagnostic method.

In another of its aspects, the present invention relates to a method for preparation of a composition as described herein, wherein the method comprises the following steps:
(i) introduction of the wild-type sequence into a plasmid or provision of a wild type genomic DNA (gDNA),
(ii) introduction of the target sequence(s) into a separate plasmid,
(iii) absolute quantification of the plasmids and/or gDNA
(iv) preparing a composition of (i) and (ii) at a defined molar ratio.

Steps (i) and (ii) of said method may be performed by any method known to the person skilled in the art for introducing a sequence into a plasmid or for providing genomic DNA.

Step (iii) includes the absolute quantification of the plasmids comprising at least one wild-type sequence, the gDNA and/or the plasmids carrying the target sequence(s). In one embodiment, step (iii) includes the absolute quantification of the plasmids comprising at least one wild-type sequence and the plasmids carrying the target sequence(s). In another embodiment, step (iii) includes the absolute quantification of the gDNA and the plasmids carrying the target sequence(s).

The absolute quantification performed in step (iii) may be performed by using any quantitative detection method known in the art, such as qPCR, dPCR or NGS. In one embodiment of the invention, the absolute quantification is performed by dPCR, in particular by ddPCR.

Based on the results obtained in step (iii), the composition of (i) and (ii) may be mixed in such a way to achieve the desired molar ratio of (ii) to (i) in a further step (iv) of the method described herein. In one embodiment of the invention, the compositions of (i) and (ii) are mixed in such a way that a molar ratio of 1:19 of the plasmid comprising the target sequence and the plasmids comprising the at least one wild-type sequence or the gDNA is obtained.

The composition obtained by the above described method may be any composition as described herein and, accordingly, may be used for any purpose as described herein.

In a further aspect of the invention kits are provided comprising the composition as described herein. These kits may be kits which include the composition as described herein as a reference composition. Alternatively, these kits may be kits which include the composition as described herein as a positive control.

In one embodiment, the kits further comprise additional reagents for the diagnostic method to be performed, e.g. for the semi-quantitative and/or quantitative mutation detection method. In one embodiment, the kits further comprise additional reagents for next generation sequencing methods and/or digital PCR. The additional reagents may include chemical reagents. Furthermore, the kits according to the invention may also comprise an instruction leaflet, etc.

All definitions, as defined and used herein, should be understood to control over dictionary definitions, definitions in documents incorporated by reference, and/or ordinary meanings of the defined terms. All patents and publications mentioned herein are incorporated by reference in their entireties.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains.

The following example is put forth so as to provide those of ordinary skill in the art with a complete disclosure and description of how to make and use the compositions of the invention. The example is intended as non-limiting example of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### Examples

Method:

Briefly, the wild type gene amplicon sequences were introduced into plasmid pUC-57. The mutated gene sequences (i.e. the particular target sequences) were introduced into separate plasmids. Several target sequences with certain distance apart were introduced into the same gene amplicon sequence and into one plasmid as can be derived from the table depicted in FIG. 1 describing the various plasmid pools comprising different combinations of target sequences. As the plasmids comprising the wild-type sequence (WT plasmids) and the plasmids carrying the target sequence(s) (Mut plasmid) share the same backbone sequence, primers were designed to carry out a droplet digital PCR (dd-PCR) with QX200 EvaGreen ddPCR Supermix to quantitate the absolute copy number of each plasmid. The same ddPCR method can be applied to quantitate the copy number of commercially available normal human genomic DNA (gDNA), e.g. Promega, Cat# G1521. After the absolute copy numbers of plasmids and/or normal human gDNA are measured, the Mut plasmids are mixed with either WT plasmids or normal human gDNA to any defined molar ratio. These plasmid compositions were then tested by an oncology Next Generation Sequencing (NGS) assay from Vela Diagnostics.

Summary:

It has been found that the plasmid compositions prepared by the above described method can be used as positive control or reference material for quantitative and semi-quantitative mutation detection methods such as digital PCR or next generation sequencing (NGS) to confirm that the technology can achieve a certain sensitivity (e.g. 5%) for any kind of target sequence(s) including the rare ones, which are hard to be found in clinical samples.

Results:

18 plasmid pools were generated by the above described method. The plasmid pools contained different numbers of specific target sequences ranging from 1 to 30 target sequence(s) as described in the table shown in FIG. 1. A total of 131 mutations were introduced into these 18 plasmid pools. All 18 plasmid pools were tested by NGS assay for 3-5 times and frequency of all target sequences were reported. The results are summarized in Table 2 below. These results show that when plasmid compositions prepared following the above protocol contain the Mut plasmids at 1:19 molar ratio to the WT plasmids, the target sequence frequency detected by the NGS assay is 5% on average for most of target sequences in the plasmids. The results also show that multiple mutations with certain distance apart in one gene amplicon sequence can be introduced into the same plasmid and that the target sequence frequencies detected by NGS assay were very similar, see e.g. plasmid 1 which carries five mutations in one BRAF gene amplicon sequence. In addition, the results suggested that various numbers of plasmids with different gene amplicon sequences of both wild type and mutated can be mixed into same pool to assess the sensitivity for mutations of different genes simultaneously, e.g. plasmids of carrying nine different target sequences as shown pool 1.

TABLE 2
Target sequence frequency measured by NGS assay performed
using plasmid pools mixed at the defined molar ratio of 5%.

| Plasmid | | | NGS | NGS | NGS | NGS | NGS | |
|---|---|---|---|---|---|---|---|---|
| Pool | Gene | Mutation | Run1 | Run2 | Run3 | Run4 | Run5 | Average |
| 1 | BRAF | c.1742A > G | 7.0 | 5.9 | 6.3 | 6.2 | 7.0 | 6.5 |
| | BRAF | c.1756G > A | 6.8 | 5.3 | 6.1 | 5.5 | 6.5 | 6.0 |
| | BRAF | c.1774A > G | 10.0 | 8.8 | 8.8 | 9.3 | 11.1 | 9.6 |
| | BRAF | c.1789C > G | 6.7 | 5.4 | 6.2 | 6.0 | 6.9 | 6.2 |
| | BRAF | c.1803A > C | 7.4 | 6.5 | 7.0 | 6.9 | 8.0 | 7.2 |
| | NRAS | c.180_181AC > TA | 2.4 | 2.3 | 2.6 | 2.9 | 2.6 | 2.6 |
| | NRAS | c.34G > C | 5.2 | 5.7 | 4.8 | 5.3 | 5.4 | 5.3 |
| | NRAS | c.52G > A | 5.3 | 5.3 | 5.0 | 5.3 | 5.3 | 5.2 |
| | CDKN2A | c.171_172CC > TT | 8.3 | 7.9 | 9.7 | 8.0 | 9.8 | 8.7 |
| | CDKN2A | c.205G > T | 6.5 | 7.8 | 9.4 | 7.8 | 9.5 | 8.2 |
| | MAP2K1 | c.332T > G | 5.5 | 5.0 | 5.3 | 5.2 | 5.1 | 5.2 |
| | MAP2K2 | c.361T > A | 5.5 | 5.0 | 5.2 | 5.3 | 5.1 | 5.2 |
| | FGFR3 | c.742C > T | 5.2 | 5.5 | 6.3 | 5.6 | 5.4 | 5.6 |
| | FGFR3 | c.1138G > A | 5.2 | 4.8 | 4.8 | 4.5 | 6.0 | 5.1 |
| | FGFR3 | c.1172C > A | 4.2 | 3.9 | 4.0 | 3.8 | 4.3 | 4.1 |
| | FGFR3 | c.1921G > A | 7.3 | 7.2 | 6.7 | 6.1 | 6.3 | 6.7 |
| | FGFR4 | c.1948A > G | 7.6 | 7.9 | 7.2 | 6.5 | 7.1 | 7.2 |
| | AKT3 | c.511G > A | 4.0 | 3.3 | 3.7 | 2.8 | 4.2 | 3.6 |
| | AKT3 | c.371A > T | 5.6 | 5.2 | 4.7 | 6.2 | 4.9 | 5.3 |
| | KIT | c.154G > A | 6.7 | 5.4 | 4.8 | 5.1 | 5.6 | 5.5 |
| | KIT | c.1727T > C | 7.4 | 8.0 | 6.1 | 7.2 | 6.0 | 6.9 |
| | KIT | c.1755C > T | 7.9 | 8.9 | 6.6 | 7.5 | 7.3 | 7.7 |
| | KIT | c.1924A > G | 6.1 | 6.4 | 6.4 | 6.5 | 5.8 | 6.2 |
| | KIT | c.1961T > C | 5.9 | 6.1 | 6.2 | 5.8 | 5.8 | 6.0 |
| | KIT | c.2446G > T | 2.0 | 2.4 | 2.6 | 1.8 | 3.4 | 2.5 |
| | KIT | c.2466T > A | 2.0 | 2.2 | 2.6 | 1.7 | 3.2 | 2.3 |
| | KIT | c.1651_1665del15 | 0.0 | 0.0 | 0.0 | 0.0 | 0.0 | 0.0 |
| | PIK3CA | c.1616C > G | 2.7 | 4.5 | 4.4 | 4.6 | 4.0 | 4.0 |
| | PIK3CA | c.1633G > A | 2.7 | 4.2 | 4.3 | 4.5 | 3.7 | 3.9 |
| | GNA11 | c.626A > T | 6.2 | 4.9 | 5.4 | 5.5 | 4.8 | 5.4 |
| 2 | BRAF | c.1760A > C | 4.8 | 4.0 | 5.0 | 5.0 | 6.4 | 5.0 |
| | BRAF | c.1776A > G | 4.9 | 4.1 | 4.9 | 5.0 | 6.2 | 5.0 |
| | BRAF | c.1789_1790CT > TC | 4.8 | 4.2 | 5.3 | 5.0 | 6.2 | 5.1 |
| | BRAF | c.1803A > T | 5.4 | 4.6 | 5.4 | 5.6 | 6.7 | 5.5 |
| | NRAS | c.181_182CA > AG | 3.7 | 4.1 | 3.2 | 3.7 | 3.3 | 3.6 |
| | NRAS | c.34G > T | 5.2 | 4.3 | 4.0 | 4.5 | 3.8 | 4.3 |
| | CDKN2A | c.172C > T | 5.3 | 5.1 | 5.7 | 5.2 | 6.4 | 5.5 |
| | MAP2K1 | c.362G > C | 5.2 | 3.7 | 5.1 | 4.4 | 4.7 | 4.6 |
| | FGFR3 | c.746C > G | 4.4 | 3.8 | 4.9 | 4.5 | 4.0 | 4.3 |
| | FGFR3 | c.1948A > C | 7.9 | 8.1 | 6.9 | 7.1 | 7.4 | 7.5 |
| | KIT | c.1656_1673del18 | 3.8 | 4.2 | 3.4 | 3.8 | 3.4 | 3.7 |
| | KIT | c.1735_1737delGAT | 6.4 | 7.1 | 5.5 | 6.4 | 6.7 | 6.4 |
| | KIT | c.2446G > C | 4.3 | 3.8 | 3.7 | 3.8 | 4.0 | 3.9 |
| | KIT | c.2466T > G | 4.6 | 3.9 | 3.7 | 3.9 | 4.0 | 4.0 |
| | PIK3CA | c.1624G > A | 4.4 | 3.5 | 3.4 | 3.5 | 3.9 | 3.7 |
| | PIK3CA | c.1637A > C | 4.8 | 4.0 | 3.7 | 3.7 | 3.8 | 4.0 |
| 3 | BRAF | c.1761C > G | 3.0 | 4.1 | 5.2 | 4.1 | | 4.1 |
| | BRAF | c.1781A > T | 1.9 | 2.3 | 3.6 | 2.4 | | 2.6 |
| | BRAF | c.1796C > T | 2.9 | 4.1 | 5.3 | 4.1 | | 4.1 |
| | BRAF | c.1813_1814AG > TT | 2.8 | 3.6 | 5.1 | 3.6 | | 3.8 |
| | NRAS | c.181A | 3.5 | 4.1 | 4.5 | 3.2 | | 3.8 |
| | NRAS | c.34G > A | 8.0 | 8.7 | 8.4 | 8.7 | | 8.4 |
| | CDKN2A | c.181G > T | 6.8 | 6.2 | 7.0 | 7.3 | | 6.8 |
| | MAP2K1 | c.370C > T | 3.7 | 4.5 | 3.1 | 4.0 | | 3.8 |
| | FGFR3 | c.1949A > T | 5.3 | 5.9 | 4.8 | 5.0 | | 5.2 |
| | KIT | c.1667_1672delAGTGGA | 3.3 | 3.8 | 3.5 | 2.7 | | 3.3 |
| | | c.1669_1674delTGGAAG | | | | | | |
| | KIT | c.2447A > T | 6.8 | 6.1 | 6.5 | 6.5 | | 6.5 |
| | KIT | c.2467T > G | 7.0 | 6.0 | 6.6 | 6.7 | | 6.6 |
| | PIK3CA | c.1624G > C | 5.1 | 3.7 | 3.6 | 5.1 | | 4.4 |
| | PIK3CA | c.1637A > G | 4.9 | 3.9 | 3.4 | 5.0 | | 4.3 |
| 4 | BRAF | c.1761C > A | 5.2 | 5.8 | 5.1 | | | 5.4 |
| | BRAF | c.1781A > G | 5.3 | 5.6 | 5.2 | | | 5.4 |
| | BRAF | c.1797_1799AGT > GAG | 5.4 | 5.8 | 5.1 | | | 5.4 |
| | BRAF | c.1814G > A | 5.2 | 5.7 | 5.0 | | | 5.3 |
| | NRAS | c.181C > G | 3.4 | 2.6 | 3.8 | | | 3.3 |
| | NRAS | c.35G > A | 7.4 | 8.3 | 8.2 | | | 8.0 |
| | FGFR3 | c.1949A > C | 5.9 | 5.3 | 6.0 | | | 5.7 |
| | KIT | c.1669T > A | 6.2 | 6.5 | 7.3 | | | 6.7 |
| | KIT | c.2474T > C | 7.3 | 7.2 | 6.5 | | | 7.0 |
| | PIK3CA | c.1625A > T | 3.6 | 3.8 | 5.4 | | | 4.3 |
| 5 | BRAF | c.1782T > A | 5.8 | 6.0 | 6.8 | | | 6.2 |
| | BRAF | c.1798_1799GT > AA | 4.3 | 5.0 | 5.7 | | | 5.0 |
| | NRAS | c.181_182CA > TT | 2.9 | 3.2 | 4.1 | | | 3.4 |
| | NRAS | c.35G > C | 5.1 | 5.2 | 5.0 | | | 5.1 |
| | KIT | c.1669T > C | 5.3 | 5.1 | 4.7 | | | 5.0 |
| | PIK3CA | c.1633G > C | 4.9 | 4.3 | 4.9 | | | 4.7 |
| 6 | BRAF | c.1783T > C | 5.2 | 5.4 | 5.7 | | | 5.4 |
| | BRAF | c.1798_1799GT > AG | 4.8 | 5.4 | 5.4 | | | 5.2 |
| | NRAS | c.181_183CAA > AAG | 2.7 | 2.7 | 3.2 | | | 2.9 |
| | NRAS | c.35G > T | 6.7 | 5.5 | 6.1 | | | 6.1 |
| | KIT | c.1669T > G | 7.3 | 8.2 | 8.4 | | | 8.0 |
| | PIK3CA | c.1634A > G | 0.0 | 0.0 | 0.0 | | | 0.0 |
| 7 | BRAF | c.1784T > C | 5.1 | 5.3 | 5.7 | | | 5.4 |
| | BRAF | c.1798G > A | 4.9 | 4.9 | 5.5 | | | 5.1 |
| | NRAS | c.182A > C | 4.2 | 4.2 | 4.5 | | | 4.3 |
| | NRAS | c.37G > C | 5.7 | 5.8 | 5.6 | | | 5.7 |
| | KIT | c.1669_1683del15 | 2.3 | 2.7 | 2.9 | | | 2.6 |
| | PIK3CA | c.1634A > C | 0.0 | 0.0 | 0.0 | | | 0.0 |
| 8 | BRAF | c.1785T > G | 5.9 | 5.5 | 6.5 | | | 5.9 |
| | BRAF | c.1798G > T | 5.5 | 4.9 | 5.2 | | | 5.2 |
| | NRAS | c.182A > T | 3.6 | 3.4 | 3.7 | | | 3.6 |
| | NRAS | c.37G > T | 6.2 | 6.5 | 7.8 | | | 6.8 |
| | KIT | c.1672_1680del9 | 5.7 | 6.7 | 6.7 | | | 6.4 |
| | PIK3CA | c.1635G > T | 5.1 | 3.6 | 4.2 | | | 4.3 |
| 9 | BRAF | c.1785T > A | 6.4 | 4.7 | 8.0 | | | 6.4 |
| | BRAF | c.1799_1800TG > AA | 3.3 | 3.1 | 4.2 | | | 3.5 |
| | NRAS | c.182A > G | 4.5 | 4.4 | 4.9 | | | 4.6 |
| | NRAS | c.37G > A | 5.4 | 5.2 | 5.8 | | | 5.5 |
| | KIT | c.1672_1686del15 | 2.1 | 0.0 | 1.8 | | | 1.9 |
| | | c.1673_1687del15 | | | | | | |
| | PIK3CA | c.1635G > C | 2.8 | 2.6 | 2.7 | | | 2.7 |
| 10 | BRAF | c.1786G > C | 7.0 | 6.9 | 6.9 | | | 6.9 |
| | BRAF | c.1799T > A | 6.6 | 6.4 | 6.6 | | | 6.6 |
| | NRAS | c.182_183AA > TG | 5.2 | 4.3 | 4.8 | | | 4.7 |
| | NRAS | c.38_39GT > TC | 6.6 | 6.9 | 7.0 | | | 6.8 |
| | KIT | c.1675_1677delGTT | 6.1 | 5.0 | 5.9 | | | 5.7 |
| | PIK3CA | c.1636C > A | 3.9 | 2.3 | 3.7 | | | 3.3 |
| 11 | BRAF | c.1790T > G | 6.5 | 5.2 | 5.9 | | | 5.9 |
| | NRAS | c.182_183AA > GG | 3.6 | 3.2 | 3.0 | | | 3.2 |
| | NRAS | c.38G > A | 5.1 | 5.0 | 5.3 | | | 5.1 |
| | KIT | c.1675G > A | 5.2 | 5.3 | 5.5 | | | 5.3 |
| | PIK3CA | c.1636C > G | 5.0 | 5.2 | 4.5 | | | 4.9 |
| 12 | BRAF | c.1790T > A | 4.3 | 4.1 | 3.7 | | | 4.0 |
| | NRAS | c.183A > T | 0.0 | 0.0 | 0.0 | | | 0.0 |
| | NRAS | c.38G > T | 4.8 | 5.0 | 4.4 | | | 4.7 |
| | KIT | c.1676T > A | 6.6 | 7.1 | 6.6 | | | 6.7 |
| | PIK3CA | c.1637A > T | 4.8 | 4.8 | 4.2 | | | 4.6 |
| 13 | BRAF | c.1797_1797A > TACTACG | 3.0 | 3.7 | 3.8 | | | 3.5 |
| | NRAS | c.183A > C | 3.7 | 3.7 | 3.8 | | | 3.7 |
| | NRAS | c.38G > C | 7.2 | 7.3 | 7.2 | | | 7.2 |
| | KIT | c.1676T > G | 4.9 | 5.3 | 5.3 | | | 5.2 |
| 14 | BRAF | c.1799_1800TG > AT | 3.4 | 3.6 | 2.6 | | | 3.2 |
| | KIT | c.1676T > C | 4.2 | 4.7 | 5.4 | | | 4.8 |
| 15 | BRAF | c.1799T > G | 4.3 | 4.4 | 4.3 | 4.3 | | 4.3 |
| | KIT | c.1679T > A | 4.2 | 4.6 | 5.0 | 4.6 | | 4.6 |
| 16 | BRAF | c.1799T > C | 6.7 | 6.3 | 6.1 | 7.4 | | 6.6 |
| | KIT | c.1679T > G | 3.9 | 4.5 | 4.2 | 4.2 | | 4.2 |
| 17 | BRAF | c.1801A > G | 5.4 | 5.4 | 5.7 | 6.0 | | 5.6 |
| 18 | BRAF | c.1801_1803delAAA | 3.6 | 3.2 | 3.2 | 3.7 | | 3.4 |
| | GNAQ | c.626A > T | 4.1 | 4.5 | 3.9 | 4.8 | | 4.3 |

1. A composition comprising:
(i) synthetic nucleic acid constructs comprising at least one inserted wild-type nucleic acid sequence or
(ii) a wild-type genomic DNA sequence in a defined molar ratio with
(iii) synthetic nucleic acid constructs comprising at least one inserted target nucleic acid sequence.

2. The composition according to claim 1, wherein the synthetic nucleic acid constructs (iii) comprise 1-50 inserted target sequence(s).

3. The composition according to claim 1, wherein the synthetic nucleic acid constructs (iii) comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 inserted target sequence (s).

4. The composition according to claim 1, wherein (iii) to (i) or (ii) have a molar ratio in the range from 1:10-1:30.

5. The composition according to claim 1, wherein the composition is a positive control composition or a reference composition for quantitative or semiquantitative mutation detection methods.

6. The composition according to claim 5, wherein the quantitative or semi-quantitative mutation detection method is digital PCR or next generation sequencing (NGS).

7. Use of the composition according to claim 1, as a control composition or as a reference composition in quantitative or semi-quantitative mutation detection methods.

8. Use of the composition according to claim 1, to test the sensitivity of a quantitative or semi-quantitative mutation detection method.

9. The use according to claim 8, wherein a sensitivity for one or more target sequence(s) occurring at 10%, 9%, 8%, 7%, 6%, 5%, 4% or 3% on average in the sample is to be confirmed.

10. The use according to claim 7, wherein the mutation detection method is digital PCR or next generation sequencing (NGS).

11. The use according to claim 7, wherein the quantitative or semi-quantitative mutation detection method is for detection of the presence or absence of sequences derived from a pathogen or from an oncogene.

12. A method of confirming the sensitivity of a semi-quantitative or quantitative detection method for one or more target sequence(s) comprising the following steps:
(i) providing a composition according to claim 1,
(ii) performing the semi-quantitative or quantitative detection method using said composition, and
(iii) assessing the sensitivity of the method.

13. A method for preparation of a composition according to claim 1 comprising the following steps:
(i) introduction of the wild-type sequence into a synthetic nucleic acid construct or provision of a wild type genomic DNA (gDNA),
(ii) introduction of at least one target nucleic acid sequence into a separate synthetic nucleic acid construct,
(iii) absolute quantification of the synthetic nucleic acid constructs, and/or the gDNA
(iv) preparing a composition of (i) and (ii) at a defined molar ratio.

14. The method according to claim 13, wherein the absolute quantification of the plasmids or the gDNA is performed by digital PCR (dPCR).

15. A kit comprising the composition according to claim 1.

16. The composition of claim 1, wherein the synthetic nucleic acid construct comprising at least one inserted wild-type nucleic acid sequence is a plasmid.

17. The composition of claim 2, wherein the synthetic nucleic acid construct comprises 1-30 inserted target sequence(s).

18. The composition of claim 4, wherein the molar ratio is 1:19.

19. The use according to claim 7, wherein the composition is used as a positive control composition.

20. The method according to claim 14 wherein the absolute quantification of the plasmids or the gDNA is performed by digital droplet PCR
